(19) United States
(12) Patent Application Publication
(10) Pub. No.: US 2023/0241237 A1
(43) Pub. Date: Aug. 3, 2023

(54) Immune Cell Redirecting Compositions and Therapeutic Uses Thereof

(71) Applicants: Emory University, Atlanta, GA (US); Children's Healthcare of Atlanta, Inc., Atlanta, GA (US)

(72) Inventors: Erik Dreaden, Atlanta, GA (US); Priscilla Do, Atlanta, GA (US); Curtis James Henry, Atlanta, GA (US); Christopher Clayton Porter, Atlanta, GA (US)

(21) Appl. No.: 17/919,481

(22) PCT Filed: Apr. 16, 2021
(86) PCT No.: PCT/US21/27778

§ 371 Date: Oct. 17, 2022

Related U.S. Application Data
(60) Provisional application No. 63/011,026, filed on Apr. 16, 2020.

(51) Int. Cl. A61K 47/68; A61K 47/69; A61P 35/00

(52) U.S. Cl. CPC A61K 47/6849; A61K 47/6929; A61K 47/6813; A61P 35/00

(57) ABSTRACT

This disclosure relates to methods for identifying molecular arrangements useful in managing diseases or conditions. In certain embodiments, this disclosure relates to treating an immune regulated disease comprising administering to a subject in need thereof an effective amount of a grouped molecular arrangement comprising a specific binding agent to CD19, a specific binding agent to CD3, and IL-12. In certain embodiments, this disclosure relates to screening test compounds comprising providing a library of multifunctional binding specificities and contacting the library with cells to evaluate in vitro therapeutic potential.

### CROSS-REFERENCE TO RELATED APPLICATIONS

This application claims the benefit of U.S. Provisional Application No. 63/011,026 filed Apr. 15, 2020. The entirety of this application is hereby incorporated by reference for all purposes.

### STATEMENT REGARDING FEDERALLY SPONSORED RESEARCH OR DEVELOPMENT

This invention was made with government support under W81XWH-19-1-0420 awarded by the Department of Defense. The government has certain rights in the invention.

### BACKGROUND

BLINCYTO® (blinatumomab) is a bispecific CD19-directed CD3 T-cell engager approved by the FDA for the treatment of relapsed or refractory CD19-positive B-cell precursor acute lymphoblastic leukemia (ALL). While response rates to blinatumomab are often impressive, remissions are not always durable. Treatment has neurological toxicities and potentially life-threatening side effects. Thus, improved therapies are needed.

Goplen report IL-12 signals through the TCR to support CD8 innate immune responses. J. Immunol. 2016, 197 (6), 2434-2443.

Berraondol et al. report interleukin-12 as a cancer immunotherapy agent. Clin Cancer Res, 2018, 24(12), 2716-8.

Rabe et al. report IL-12 abrogates calcineurin-dependent immune evasion during leukemia progression. Cancer Res, 2019, 79(14): 3702-3713.

Momin et al. report anchoring of intratumorally administered cytokines to collagen safely potentiates systemic cancer immunotherapy. Sci. Transl. Med. 2019, 11 (498), eaaw2614.

References cited herein are not an admission of prior art.

### SUMMARY

This disclosure relates to methods for identify molecular arrangements useful in managing diseases or conditions. In certain embodiments, this disclosure relates to treating an immune regulated disease comprising administering to a subject in need thereof an effective amount of a grouped molecular arrangement comprising a specific binding agent to CD19, a specific binding agent to CD3, and IL-12. In certain embodiments, this disclosure relates to screening test compounds comprising providing a library of multifunctional binding specificities and contacting the library with cells to evaluate in vitro therapeutic potential.

In certain embodiments, this disclosure relates to a drug discovery platform for identifying multi-functional therapeutics, e.g., bi-functional, tri-functional, quad-functional, etc., which facilitate the interaction of immune cells with diseased target cells and cytokine proteins.

In certain embodiments, this disclosure relates to a screening method comprising providing variant relative abundance of T cell, B cell, and IL-12 antibody binding domains on the surface of molecular arrangements or nanoparticles and culturing the molecular arrangements or nanoparticles in the presence of human T cells and leukemic B cells. Typically, the molecular arrangements or particles are at a concentration of about 100 to 150 pM, e.g., 130 pM for more than 1, 2, 4, 8, 16, 24, or 72 h at a 1:1 effector-to-target cell ratio (T cell to B cells ratio). Typically, the method includes measuring B cell lysis and/or T cell proliferation via flow cytometry and imaging.

In certain embodiments, this disclosure contemplates a multi-functional therapeutic comprising (i) a particle core and (ii) a linker which binds surface-displayed ligands with affinity towards (iii) T cells, (iv) B cells, and (v) cytokines.

In certain embodiments, the particle cores are magnetic nanoparticles, e.g., iron oxide (30-120 nm diameter), which display an immunoglobulin-binding protein, e.g., Protein G, that is capable of binding the Fc portion of IgG antibodies wherein the nanoparticles are coated with antibodies that specifically bind CD3 epsilon chain, a component of the T cell receptor, as well as antibodies that specifically bind CD19, a lineage marker for B cells, and antibodies that specifically bind, in a non-neutralizing manner, the cytokine, IL-12.

In certain embodiments, the linage marker for B cells is CD19, CD20, or CD22.

In certain embodiments, the conjugation of specific binding agents or antibodies is through use of a covalent linker, e.g., a maleimide linker.

In certain embodiments, the trifunctional particle can induce the specific lysis of B cells by T cells and that concurrent stimulation of T cells with IL-12 can further improve the extent of this lysis, in part, due to T cell proliferation.

In certain embodiments, it is contemplated that the particles and molecular arrangements can be used in the treatment of B cell cancers, immune regulated diseases, and autoimmune diseases (e.g. lupus) driven by auto-antibody production by B cells.

In certain embodiments, the immune regulated disease is cancer, a chronic viral infection, or lupus. In certain embodiments, the immune regulated disease is a hematological cancer such as leukemia. In certain embodiments, the molecular arrangement is a nanoparticle, cell, liposome, virus-like particle (VLP), self-assembled protein cage, fusion protein, recombinant vector, or nucleic acid encoding the self-assembled protein or fusion protein. In certain embodiments, the molecular arrangement is linear or branched polymers, linear or branched peptides, or dendrimers.

In certain embodiments, this disclosure relates to methods of treating immune regulated diseases comprising administering to a subject in need thereof an effective amount of a grouped molecular arrangement comprising a specific binding agent to CD19 and a specific binding agent to CD3 in combination with an IL-12 sequence, or vector or nucleic acid encoding a protein thereof. In certain embodiments, the immune regulated disease is cancer, chronic viral infection, or lupus. In certain embodiments, the immune regulated disease is a hematological cancer such as leukemia. In certain embodiments, the grouped molecular arrangement or nanoparticle is administered in combination with another anticancer agent.

In certain embodiments, this disclosure relates to methods of treating an immune regulated disease comprising administering to a subject in need thereof an effective amount of a nanoparticle comprising on the surface of the nanoparticle a specific binding agent to CD19, a specific binding agent to CD3, and IL-12. In certain embodiments, the immune regulated disease is cancer, chronic viral infection, or lupus.

In certain embodiments, the nanoparticle comprises on the surface a specific binding agent to an antigen sequence, wherein the specific binding agent to the antigen is specifically binding the antigen sequence conjugated to IL-12 comprising the antigen.

In certain embodiments, the nanoparticle comprises on the surface a specific binding agent to an IL-12 sequence, wherein the specific binding agent to the IL-12 sequence is specifically binding IL-12.

In certain embodiments, the nanoparticle has a hydrodynamic diameter of less than 150 nm or 100 nm and comprises less than 5 specific binding agents to CD19 on the surface of the particle.

In certain embodiments, the nanoparticle has a hydrodynamic diameter of less than 150 nm or 100 nm and comprises between 20 to 70 specific binding agents to CD3 on the surface of the particle.

In certain embodiments, the nanoparticle has a ratio of specific binding agents to CD3 compared to specific binding agents to CD19 which is in excess of 10-fold.

In certain embodiments, the nanoparticle has a ratio of specific binding agents to CD3 compared to specific binding agents to CD19 which is in excess of 20-fold.

In certain embodiments, the individual nanoparticles each have a hydrodynamic size of less than 200 nm, have 25 to 60 specific binding agents to CD3, 2 to 4 specific binding agents to CD19, and 1 to 12 interleukin-12 sequences on the surface of the nanoparticle.

In certain embodiments, the specific binding agents to CD3 are antibodies.

In certain embodiments, the specific binding agents to CD19 are antibodies.

In certain embodiments, this disclosure relates to pharmaceutical compositions comprising a particle or molecular arrangement disclosed herein.

In certain embodiments, this disclosure relates to pharmaceutical compositions comprising a nanoparticle comprising on the surface of the nanoparticle a specific binding agent to CD19, a specific binding agent to CD3, and IL-12.

In certain embodiments, a molecular arrangement, nanoparticle, polypeptide, vector, or nucleic acid disclosed herein comprise or are conjugated to a label.

In certain embodiments, this disclosure relates to the production of a medicament comprising a composition disclosed herein for use in the treatment of a disease or conditions disclosed herein.

### BRIEF DESCRIPTION OF THE SEVERAL VIEWS OF THE DRAWINGS

FIGS. 1A-1D show data indicating recombinant IL-12 enhances activity of the bispecific T cell engager therapy, blinatumomab (blina).

FIG. 1A illustrates assay conditions for the coculture of primary human CD8+ T cells with CD19+ NALM-6 leukemia cells.

FIG. 1B shows data on blinatumomab-induced lysis of NALM-6 leukemia cells.

FIG. 1C shows data on T cell proliferation enhanced by coincubation with IL-12 as measured by flow cytometry represented as dye-dilution histograms. Cocultures in were treated with blina (7 ng/mL) with or without IL-12 (3.5 ng/mL) in comparison to PBS vehicle over 72 h.

FIG. 1D data indicates blinatumomab and IL-12 synergize to enhance T cell activation as measured by ELISA of IFNγ secretion into coculture supernatants. Cocultures in were treated with blina (7 ng/mL) with or without IL-12 (3.5 ng/mL) in comparison to PBS vehicle over 48 h.

FIG. 2 illustrates the structure and assembly of bispecific T cell engaging cytokines (BiTEokines). Schematic of drug-induced synapse formation between T cells and leukemic B cells, as well as synapse-targeted delivery of the cytokine, IL-12. Inset illustrates the modular and rapid self-assembly of CD19×CD3×IL12 BiTEokines via addition of human IgG to protein G-conjugated iron-oxide nanoparticles and subsequent cytokine complexation. The solid arrow denotes cytokine release or trans presentation.

FIG. 3A illustrate of test compound library assembly. High-throughput assembly and screening enables rapid identification of BiTEokines that induce efficient leukemia cell lysis.

FIG. 3B illustrates the structural diversity of the BiTEokine library.

FIG. 3C shows size data associated with a representative test compound as measured by antibody fluorescence intensity, dynamic light scattering, and transmission electron microscopy.

FIG. 3D shows data on parallel screening results rank-ordered by drug-induced lysis of CD19+ NALM-6 leukemia cells by primary human CD8+ T cells.

FIG. 4 shows data indicating CD19×CD3×IL12 BiTEokines bind specifically and induce efficient leukemia cell lysis. Cell fluorescence from various BiTEokine antibodies observed in cocultures gated on (top) primary human T cells or (bottom) NALM-6 leukemia cells as measured by flow cytometry. Top left: CD8+ T cells exhibit CD3 antibody fluorescence that increases in intensity with relative abundance on BiTEokines. Bottom middle: CD19+ leukemia cells exhibit CD19 antibody fluorescence that increases in intensity with relative abundance on BiTEokines.

FIG. 5 shows images indicating BiTEokines localize at the interface between primary human T cells and NALM-6 leukemic B cells. Imaging flow cytometry of CD8+ T cells cocultured with NALM-6 leukemia cells at a 1:1 E:T ratio and treated with (top) blinatumomab or (bottom) CD19×CD3×IL12 BiTEokines (35) for 24 h at equimolar concentrations (130 pM). Report images from two different T cell donors. Arrowheads indicate localization of BiTEokines at the T-B cell interface. E:T, effector-to-target cell ratio. Scale bar is 7 μm.

FIG. 6 shows data on particle coverage hit compound #032 and #035 compared with a maleimide-conjugated analog.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow reference will be made to a number of terms that shall be defined to have the following meanings unless a contrary intention is apparent.

A “certain embodiment” refers to a specific or contemplate example but is not necessarily limited to such an example.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods.

CD3 (cluster of differentiation 3) is a protein complex of several protein chains that are involved in activating cytotoxic T cells (CD8+ naive T cells) and T helper cells (CD4+ naive T cells). It is contemplated that specific binding agents may bind to the CD3 alpha, beta, delta, gamma, epsilon, or zeta chain or combinations thereof. Experimental data indicates that particles with antibodies targeting the interface of both CD3 epsilon and gamma are therapeutically active. It is contemplated that binding exclusively with, or in addition to, the other extracellular CD3 chains (delta, alpha, beta) would similarly function in inducing lysis by T cells. Reports indicate that mutations CD3 gamma may contribute to the increased rate of autoimmunity. The human CD3 gamma chain precursor sequence is NCBI Reference Sequence: NP_000064.1. The CD3 epsilon chain plays an essential role in correct T-cell development. Initiates the TCR-CD3 complex assembly by forming the two heterodimers CD3delta/CD3epsilon and CD3gamma/CD3epsilon. The human reference sequence for CD3 epsilon chain precursor is NCBI Reference Sequence: NP_000724.1. The CD3 zeta chain plays a role in coupling antigen recognition. Low expression of the zeta results in impaired immune response. The human references sequence for CD3 T-cell receptor zeta chain is known as NCBI Reference Sequence: NP_932170.1.

CD19 is a protein typically found on the surface of B cells. The human references sequence for B-lymphocyte antigen CD19 isoform 1 precursor is NCBI Reference Sequence: NP_001171569.1. Amino acids 1-20 are a signal sequence. Amino acids 21-291 are the extracellular domain. Amino acids 292-313 are a transmembrane region.

CD20 is B-lymphocyte surface molecule which plays a role in the development and differentiation of B-cells into plasma cells. The human reference sequence for CD20 is NCBI Reference Sequence: NP 690606.1.

CD22 is a is B-lymphocyte surface molecule which involved in the regulation of innate and adaptive B cell responses and autoimmunity. The CD22 gene expresses 5 known isoforms. The longest human CD22 isoform 1 precursor sequence is NCBI Reference Sequence: NP_001762.2.

Interleukin-12 (IL-12) also known as IL-12p70 is a disulfide linked heterodimeric (IL12-alpha or p35 and IL12-beta or p40) cytokine that induces the production of interferon-γ (IFN-γ). The human reference sequence for IL-12 alpha isoform 1 precursor is NCBI Reference Sequence: NP_000873.2. The human reference sequence for interleukin-12 subunit beta precursor is NCBI Reference Sequence: NP 002178.2.

“Subject” refers to any animal, preferably a human patient, livestock, rodent, monkey or domestic pet.

“Cancer” refers any of various cellular diseases with malignant neoplasms characterized by the proliferation of cells. Cancer may or may not be present as a tumor mass with a defined boundary. It is not intended that the diseased cells must actually invade surrounding tissue and metastasize to new body sites. Cancer can involve any tissue of the body and have many different forms in each body area. Within the context of certain embodiments, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, HER2 for breast cancer, or others.

“Hematological cancer” refers to a cancer that begins in blood-forming tissue, such as the bone marrow, or in the cells of the immune system. Examples of hematologic cancer are acute lymphoblastic leukemia, chronic lymphocytic leukemia, lymphoma, Hodgkin lymphoma, multiple myeloma, acute myelogenous leukemia, chronic myelogenous leukemia, myelodysplastic syndromes, myeloproliferative neoplasms, essential thrombocythemia, polycythemia vera, and myelofibrosis.

“Prevent” and “preventing” include the prevention of the recurrence, spread or onset. It is not intended that the present disclosure be limited to complete prevention. In some embodiments, the onset is delayed, or the severity is reduced.

“Treat” and “treating” are not limited to the case where the subject (e.g., patient) is cured and the disease is eradicated. Rather, embodiments, of the present disclosure also contemplate treatment that merely reduces symptoms, and/or delays disease progression.

The terms “in combination with” when used to describe administration with an additional treatment mean that the agent may be administered prior to, together with, or after the additional treatment, or a combination thereof.

The terms “effective amount” refer to that amount of a compound or pharmaceutical composition described herein that is sufficient to effect the intended application including, but not limited to, disease treatment, as illustrated below. In relation to a combination therapy, an “effective amount” indicates the combination of agent results in synergistic or additive effect when compared to the agents individually. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific dose will vary depending on, for example, the particular compounds chosen, the dosing regimen to be followed, whether it is administered in combination with other agents, timing of administration, the tissue to which it is administered, and the physical delivery system in which it is carried.

A “chemotherapy agent,” “chemotherapeutic,” “anti-cancer agent,” or the like, refer to molecules that are recognized to aid in the treatment of a cancer. Contemplated examples include the following molecules or derivatives such as abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cab ozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelalisib, imatinib, talimogene laherparepvec, ipilimumab, romidepsin, ixabepilone, ixazomib, ruxolitinib, cabazitaxel, palifermin, pembrolizumab, ribociclib, ti sagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, zoledronic acid, or combinations thereof such as cyclophosphamide, methotrexate, 5-fluorouracil (CMF); doxorubicin, cyclophosphamide (AC); mustine, vincristine, procarbazine, prednisolone (MOPP); sdriamycin, bleomycin, vinblastine, dacarbazine (ABVD); cyclophosphamide, doxorubicin, vincristine, prednisolone (CHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC).

The terms “protein,” “peptide,” and “polypeptide” refer to compounds comprising amino acids joined via peptide bonds and are used interchangeably. As used herein, where “amino acid sequence” is recited herein to refer to an amino acid sequence of a protein molecule. An “amino acid sequence” can be deduced from the nucleic acid sequence encoding the protein. However, terms such as “polypeptide” or “protein” are not meant to limit be limited to natural amino acids. The term includes non-naturally occurring amino acids and modifications such as, substitutions, glycosylations, and addition of hydrophilic or lipophilic moieties.

In the context of a fusion or chimeric peptide (a peptide comprising two or more peptide segments), a “heterologous” peptide sequence is a comparative term and refers to a peptide segment that would not naturally occur together with the other segment, e.g., because one the of the segments is derived from a different organism, a label, or random. In certain embodiments, a heterologous fusion peptide of this disclosure may contain a peptide sequence disclosed herein and a fluorescent protein sequence, a protease cleaving sequence, a self-cleaving sequence, a ligand, antibody epitope, or a polyhistidine sequence.

As used herein, the term “conjugated” refers to linking molecular entities through covalent bonds, or by other specific binding interactions, such as due to hydrogen bonding and other van der Walls forces. The force to break a covalent bond is high, e.g., about 1500 pN for a carbon to carbon bond. The force to break a combination of strong protein interactions is typically a magnitude less, e.g., biotin to streptavidin is about 150 pN. Thus, a skilled artisan would understand that conjugation must be strong enough to bind molecular entities in order to implement the intended results.

A “linking group” refers to any variety of molecular arrangements that can be used to bridge to molecular moieties together. An example formula may be —Rₙ— wherein R is selected individually and independently at each occurrence as: —CRₙRₙ—, —CHRₙ—, —CH—, —C—, —CH₂—, —C(OH)Rₙ, —C(OH)(OH)—, —C(OH)H, —C(Hal)Rₙ—, —C(Hal)(Hal)-, —C(Hal)-, —C(N₃)Rₙ—, —C(CN)Rₙ—, —C(CN)(CN)—, —C(CN)H—, —C(N₃)(N₃)—, —C(N₃)H—, —O—, —S—, —N—, —NH—, —NRₙ—, —(C═O)—, —(C═NH)—, —(C═S)—, —(C═CH₂)—, which may contain single, double, or triple bonds individually and independently between the R groups. If an R is branched with an Rₙ it may be terminated with a group such as —CH₃, —H, —CH═CH₂, —CCH, —OH, —SH, —NH₂, —N₃, —CN, or -Hal, or two branched Rs may form an aromatic or non-aromatic cyclic structure. It is contemplated that in certain instances, the total Rs or “n” may be less than 100 or 50 or 25 or 10. Examples of linking groups include bridging alkyl groups and alkoxyalkyl groups.

A “ligand” refers to any organic molecule, i.e., substantially comprised of carbon, hydrogen, and oxygen, that specifically binds to a “receptor.” Receptors are organic molecules typically found on the surface of a cell. Through binding a ligand to a receptor, the cell has a signal of the extra cellular environment which may cause changes inside the cell. As a convention, a ligand is usually used to refer to the smaller of the binding partners from a size standpoint, and a receptor is usually used to refer to a molecule that spatially surrounds the ligand or portion thereof. However as used herein, the terms can be used interchangeably as they generally refer to molecules that are specific binding partners. For example, a glycan may be expressed on a cell surface glycoprotein and a lectin protein may bind the glycan. As the glycan is typically smaller and surrounded by the lectin protein during binding, it may be considered a ligand even though it is a receptor of the lectin binding signal on the cell surface. An antibody may be a receptor, and the epitope may be considered the ligand. In certain embodiments, a ligand is contemplated to be a compound that has a molecular weight of less than 500 or 1,000. In certain embodiments, a receptor is contemplated to be a proteinaceous compound that has a molecular weight of greater than 1,000, 2,000 or 5,000.

A “label” refers to a detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a peptide “label” refers to incorporation of a heterologous polypeptide in the peptide, wherein the heterologous sequence can be identified by a specific binding agent, antibody, or bind to a metal such as nickel/nitrilotriacetic acid, e.g., a poly-histidine sequence. Specific binding agents and metals can be conjugated to solid surfaces to facilitate purification methods. A label includes the incorporation of a radiolabeled amino acid or the covalent attachment of biotinyl moieties to a polypeptide that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionucleotides (such as ³⁵S or ¹³¹I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels may be attached by spacer arms of various lengths to reduce potential steric hindrance.

A “radionuclide” or “radioactive isotope” refers to molecules of enriched isotopes that exhibit radioactive decay (e.g., emitting positrons). Such isotopes are also referred to in the art as radioisotopes, e.g., a naturally occurring isotopes that exhibit radioactive decay with an isotope distribution that is enriched, e.g., is several fold greater than natural abundance. In certain embodiments, is contemplated that the radionuclides are limited to those with a half live of less than 1 hour and those with a half-life of more than 1 hour but less than 24 hours. Radioactive isotopes are named herein using various commonly used combinations of the name or symbol of the element and its mass number (e.g., ¹⁸F, F-18, or fluorine-18).

The term “specific binding agent” refers to a molecule, such as a proteinaceous molecule, that binds a target molecule with a greater affinity than other random molecules or proteins. Examples of specific binding agents include antibodies that bind an epitope of an antigen or a receptor which binds a ligand. “Specifically binds” refers to the ability of a specific binding agent (such as an ligand, receptor, enzyme, antibody or binding region/fragment thereof) to recognize and bind a target molecule or polypeptide, such that its affinity (as determined by, e.g., affinity ELISA or other assays) is at least 10 times as great, but optionally 50 times as great, 100, 250 or 500 times as great, or even at least 1000 times as great as the affinity of the same for any other or other random molecule or polypeptide.

In certain contexts, an “antibody” refers to a protein based molecule that is naturally produced by animals in response to the presence of a protein or other molecule or that is not recognized by the animal's immune system to be a “self” molecule, i.e. recognized by the animal to be a foreign molecule and an antigen to the antibody. The immune system of the animal will create an antibody to specifically bind the antigen, and thereby targeting the antigen for elimination or degradation. It is well recognized by skilled artisans that the molecular structure of a natural antibody can be synthesized and altered by laboratory techniques. Recombinant engineering can be used to generate fully synthetic antibodies or fragments thereof providing control over variations of the amino acid sequences of the antibody. Thus, as used herein the term “antibody” is intended to include natural antibodies, monoclonal antibody, or non-naturally produced synthetic antibodies, and binding fragments, such as single chain binding fragments. These antibodies may have chemical modifications. The term “monoclonal antibodies” refers to a collection of antibodies encoded by the same nucleic acid molecule that are optionally produced by a single hybridoma (or clone thereof) or other cell line, or by a transgenic mammal such that each monoclonal antibody will typically recognize the same antigen. The term “monoclonal” is not limited to any particular method for making the antibody, nor is the term limited to antibodies produced in a particular species, e.g., mouse, rat, etc.

From a structural standpoint, an antibody is a combination of proteins: two heavy chain proteins and two light chain proteins. The heavy chains are longer than the light chains. The two heavy chains typically have the same amino acid sequence. Similarly, the two light chains have the same amino acid sequence. Each of the heavy and light chains contain a variable segment that contains amino acid sequences which participate in binding to the antigen. The variable segments of the heavy chain do not have the same amino acid sequences as the light chains. The variable segments are often referred to as the antigen binding domains. The antigen and the variable regions of the antibody may physically interact with each other at specific smaller segments of an antigen often referred to as the “epitope.” Epitopes usually consist of surface groupings of molecules, for example, amino acids or carbohydrates. The terms “variable region,” “antigen binding domain,” and “antigen binding region” refer to that portion of the antibody molecule which contains the amino acid residues that interact with an antigen and confer on the antibody its specificity and affinity for the antigen. Small binding regions within the antigen-binding domain that typically interact with the epitope are also commonly alternatively referred to as the “complementarity-determining regions, or CDRs.”

Hydrophilic polymers contain polar or charged functional groups, rendering them soluble in water. Examples include polyethylene glycol, polylactides, polyglycolide, poly(ε-caprolactone), poly(2-methoxyethyl acrylate), poly(tetrahydrofurfuryl acrylate), poly(2-methacryloyloxyethyl phosphorylcholine), poly(p-dioxanone), poly(serine methacrylate), poly [oligo(ethylene glycol) vinyl ether], poly {[2-(methacryloyloxy)ethyl], copolymers of ethylene glycol and propylene glycol, poly(oxyethylated polyol), poly(olefmic alcohol), poly(vinylpyrrolidone), poly(hydroxyalkylmethacrylamide), poly(hydroxyalkylmethacrylate), poly(saccharides), poly(alpha-hydroxy acid), and poly(vinyl alcohol). “PEG,” “polyethylene glycol” and “poly(ethylene glycol)” refers to water-soluble poly(ethylene oxide). Typically, PEGs comprise the following structure “—(OCH₂CH₂)ₙ—” where (n) is 2 to 4000.

The term “recombinant” when made in reference to a nucleic acid molecule refers to a nucleic acid molecule which is comprised of segments of nucleic acid joined together by means of molecular biological techniques provided that the entire nucleic acid sequence does not occurring in nature, i.e., there is at least one mutation in the overall sequence such that the entire sequence is not naturally occurring even though separately segments may occurring in nature. The segments may be joined in an altered arrangement such that the entire nucleic acid sequence from start to finish does not naturally occur. The term “recombinant” when made in reference to a protein or a polypeptide refers to a protein molecule that is expressed using a recombinant nucleic acid molecule.

The terms “vector” or “expression vector” refer to a recombinant nucleic acid containing a desired coding sequence and appropriate nucleic acid sequences necessary for the expression of the operably linked coding sequence in a particular host organism or expression system, e.g., cellular or cell-free. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

Protein “expression systems” refer to in vivo and in vitro (cell free) systems. Systems for recombinant protein expression typically utilize cells (somatic cells) transfecting with a DNA expression vector that contains the template. The cells are cultured under conditions such that they translate the desired protein. Expressed proteins are extracted for subsequent purification. In vivo protein expression systems using prokaryotic and eukaryotic cells are well known. Proteins may be recovered using denaturants and protein-refolding procedures. In vitro (cell-free) protein expression systems typically use translation-compatible extracts of whole cells or compositions that contain components sufficient for transcription, translation, and optionally post-translational modifications such as RNA polymerase, regulatory protein factors, transcription factors, ribosomes, tRNA cofactors, amino acids and nucleotides. In the presence of an expression vectors, these extracts and components can synthesize proteins of interest. Cell-free systems typically do not contain proteases and enable labeling of the protein with modified amino acids. Some cell free systems incorporated encoded components for translation into the expression vector. See, e.g., Shimizu et al., Cell-free translation reconstituted with purified components, 2001, Nat. Biotechnol., 19, 751-755 and Asahara & Chong, Nucleic Acids Research, 2010, 38(13): e141, both hereby incorporated by reference in their entirety.

### Molecular Arrangements and Uses for Identifying Optimal Immune Stimulating Therapies

In certain embodiments, the molecular arrangements disclosed herein are useful for identify optimal immune stimulating therapies.

In certain embodiments, this disclosure relates to methods of screening a library of molecular arrangements for an associated therapeutic effect comprising: assembling a library of molecular arrangements, wherein individual molecular arrangements in the library comprise a first set of specific binding agents that bind to a first cell marker on found on cells associated with a target disease, wherein individual molecular arrangements in the library comprise a second set of specific binding agents that bind to a second cell marker found on cells associated with immune cells that kill cells associated with a target disease, and wherein individual molecular arrangements in the library comprise a set of ligands that bind to a receptor found on immune cells that kill or lyse cells associated with a target disease; contacting the library of molecular arrangements with immune cells and cells associated with a target disease; and detecting the associated therapeutic effect of the individual molecular arrangement based on a physical property of the cells associated with a target disease.

In certain embodiments, the immune cells are neutrophils, eosinophils, basophils, mast cells, monocytes, macrophages, dendritic cells, natural killer cells, and lymphocytes (B cells and T cells). In certain embodiments, the immune cells express CD3, CD4, CD8, or combinations thereof.

In certain embodiments, the cells associated with a target disease are cancer cells.

In certain embodiments, the physical property of the cells associated with a target disease is lysis, survival, growth, or death of the disease cells when in contact with the immune cells.

In certain embodiments, the first and second set of specific binding agents are a first and second set of antibodies that specifically bind different antigens on immune cells and cells associated with a target disease, and the set of ligands are interleukins, e.g., IL-1, IL-2, IL-3, IL-4, IL-5, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36, or combinations thereof. In certain embodiments, the interleukin, e.g., IL-12, is a mutant in which the disulfide bond which typically links its two subunits is replaced by a flexible peptide sequence referred to as single-chain interleukin.

In certain embodiments, the molecular arrangements are set of nanoparticles, cells, liposomes, virus-like particles (VLPs), self-assembled protein cages, fusion proteins, recombinant vectors, or nucleic acids encoding the self-assembled proteins or fusion proteins. In certain embodiments, the molecular arrangement is linear or branched polymers, linear or branched peptides, or dendrimers.

In certain embodiments, this disclosure contemplates that the molecular arrangements are nanoparticles comprising a surface that specifically binds an Fc region (immunoglobulin IgG, or IgA, or IgM) of antibodies wherein the surface bound antibodies specifically bind two or three or more unique antigens providing antibody coated particles or antibody coated particles with antigen bound segments.

In certain embodiments, the linage marker for B cells is CD19, CD20, or CD22.

In certain embodiments, the conjugation of specific binding agents or antibodies is through use of a covalent linker, e.g., a maleimide linker.

In certain embodiments, the nanoparticles comprise a magnetic core.

In certain embodiments the molecular arrangement comprising a specific binding agent to CD19, a specific binding agent to CD3, and an IL-12 sequence, or vector or nucleic acid encoding a fusion protein thereof.

In certain embodiments, antibody coated particles are contacted with polypeptide sequences, interleukins, or antigens conjugates for immobilization of the polypeptide, interleukins, antigen conjugates to the surface of the particles by a portion of antibodies coated on the particles.

In certain embodiments, the libraries comprise mixtures of antibodies on the surface of particles each with variant concentrations at the surface.

In certain embodiments, the libraries comprise mixtures of 1) first antibodies, 2) second antibodies, and 3) antibody bound polypeptides, interleukins, or antigen conjugates.

In certain embodiments, the first antibody on the surface of the nanoparticle has a molecular ratio of greater than 1 compared to the second antibody on the surface of the particle.

In certain embodiments, the first antibody on the surface of the nanoparticle has a molecular ratio of greater than 2 compared to the second antibody on the surface of the particle.

In certain embodiments, the first antibody on the surface of the nanoparticle has a molecular ratio of greater than 5 compared to the second antibody on the surface of the particle.

In certain embodiments, the first antibody on the surface of the nanoparticle has a molecular ratio of greater than 1 compared to antibody bound polypeptides, interleukins, or antigen conjugates on the surface of the particle.

In certain embodiments, the first antibody on the surface of the nanoparticle has a molecular ratio of greater than 2 compared to antibody bound polypeptides, interleukins, or antigen conjugates on the surface of the particle.

In certain embodiments, a first antibody on the surface of the nanoparticle has a molecular ratio of greater than 5 compared to antibody bound polypeptides, interleukins, or antigen conjugates on the surface of the particle.

In certain embodiments, the first antibody on the surface of the nanoparticle has a molecular ratio of less than 1 compared to antibody bound polypeptides, interleukins, or antigen conjugates on the surface of the particle.

In certain embodiments, a first antibody on the surface of the nanoparticle has a molecular ratio of less than 2 compared to antibody bound polypeptides, interleukins, or antigen conjugates on the surface of the particle.

In certain embodiments, a first antibody on the surface of the nanoparticle has a molecular ratio of less than 5 compared to antibody bound polypeptides, interleukins, or antigen conjugates on the surface of the particle.

In certain embodiments, this disclosure relates to method of identing molecular arrangements for treating immune regulated disorders. In certain embodiments, the method comprises generating a library of molecular arrangements that specifically bind two or three or more unique cell surface markers, wherein the library of molecular arrangements comprises different concentrations of the specific binding agents that bind the unique cells surface marker. In certain embodiments, the molecular arrangements comprise surface proteins, e.g., cytokines or interleukins.

In certain embodiments, the molecular arrangements are nanoparticles comprising antibodies that bind cell surface markers, optionally the nanoparticles comprise surface proteins, e.g. surface bound interleukins, IL-12, with variant surface concentrations or coverage on different nanoparticles in the library.

In certain embodiments, the molecular arrangements or nanoparticles are contacted with an immune cell or disease cell and the cell(s) are evaluated for growth, lysis, death, genetic expression, mRNA expression, secretions, or other phenotypic changes. In certain embodiments, the cells are sample cells, cancerous cells, T cells, or natural killer cells. In certain embodiments, the cells express CD3, CD4, and/or CD8.

### Pharmaceutical Methods of Use

In certain embodiments, this disclosure relates to methods of treating immune regulated diseases comprising administering to a subject in need thereof an effective amount of a grouped molecular arrangement comprising a specific binding agent to CD19, a specific binding agent to CD3, and an IL-12 sequence, or vector or nucleic acid encoding a fusion protein thereof. In certain embodiments, the immune regulated disease is cancer, chronic viral infection, or lupus. In certain embodiments, the immune regulated disease is a hematological cancer such as leukemia.

In certain embodiments, this disclosure relates to methods of treating immune regulated diseases comprising administering to a subject in need thereof an effective amount of a grouped molecular arrangement comprising a specific binding agent to CD19 and a specific binding agent to CD3 in combination with an IL-12 sequence, or vector or nucleic acid encoding a protein or fusion thereof.

In certain embodiments, the immune regulated disease is a hematological cancer such as leukemia.

In certain embodiments, the molecular arrangement is on the surface of a nanoparticle or cell or a multispecific immunoglobulin construct.

In certain embodiments, the multispecific immunoglobulin construct is IL-12 conjugated to blinatumomab.

In certain embodiments, this disclosure relates to methods of treating cancer or immune regulated disease comprising administering to a subject in need thereof an effective amount of a molecular arrangement comprising a specific binding agent to CD19 conjugated to a specific binding agent to CD3 in combination with administering IL-12 or a vector or nucleic acid encoding IL-12. In certain embodiments, the cancer is a hematological cancer such as leukemia.

In certain embodiments, this disclosure relates to methods of treating an immune regulated disease comprising administering to a subject in need thereof an effective amount of a molecular arrangement or nanoparticle comprising on the surface a specific binding agent to CD19, a specific binding agent to CD3, and IL-12.

In certain embodiments, the nanoparticle comprises on the surface a specific binding agent to an IL-12 sequence, wherein the specific binding agent to the IL-12 sequence is specifically binding IL-12.

In certain embodiments, the nanoparticle comprises on the surface a specific binding agent to an antigen sequence, wherein the specific binding agent to the antigen is specifically binding the antigen sequence conjugated to an IL-12 containing polypeptide comprising the antigen.

In certain embodiments, the immune regulated disease is cancer, chronic viral infection, or lupus. In certain embodiments, the immune regulated disease is a hematological cancer such as leukemia.

In certain embodiments, the immune regulated disease is associated with a B cell depleting therapy such as autoimmune disease or B cell-dependent autoimmune diseases. In certain embodiments, the disease is cancer, infections, multiple sclerosis, relapsing multiple sclerosis, progressive multiple sclerosis, primary progressive multiple sclerosis, secondary progressive multiple sclerosis, anti-N-methyl-D-aspartate receptor (NMDAR) encephalitis, lupus, lupus nephritis, lupus erythematosus, systemic lupus erythematosus, pemphigus vulgaris, mucosal-dominant pemphigus vulgaris, rheumatoid arthritis, neuromyelitis optica spectrum disorder, thrombocytopenic purpura, idiopathic thrombocytopenic purpura, vasculitis, Sjögren syndrome, or myasthenia gravis.

In certain embodiments, any of the methods disclosed herein using molecular arrangements or particles disclosed herein may be administered or used in combination with another chemotherapy or chemotherapy agent. In certain embodiments, the chemotherapy agent is an anti-PD-1, anti-PD-L1 anti-CTLA4 antibody or combinations thereof, such as an anti-CTLA4 (e.g., ipilimumab, tremelimumab) and anti-PD1 (e.g., nivolumab, pembrolizumab, cemiplimab) and anti-PD-L1 (e.g., atezolizumab, avelumab, durvalumab).

In certain embodiments, the chemotherapy is a chimeric antigen receptor T cell therapy such as an anti-CD19 chimeric antigen receptor T cell therapy, Desmoglein 3 chimeric autoantibody receptor T cells (DSG3-CAART) expressing the pemphigus vulgaris (PV) autoantigen DSG3 fused to CD137-CD3ζ signaling domains, or Descartes-08, i.e., autologous CD8+ T cells that express anti-B cell maturation antigen chimeric antigen receptor.

In certain embodiments, the method of administration is in a subject with a lymphodepleted environment due to prior or concurrent administration of lymphodepleting agents. In certain embodiments, lymphodepleting agents are cyclophosphamide and/or fludarabine.

### Pharmaceutical Compositions

In certain embodiments, this disclosure relates to compositions comprising a grouped molecular arrangement comprising a specific binding agent to CD19, a specific binding agent to CD3, and an IL-12 sequence, or vector or nucleic acid encoding a fusion protein thereof.

In certain embodiments, this disclosure relates to pharmaceutical compositions comprising a molecular arrangement or nanoparticle disclosed herein comprising on the surface a specific binding agent to CD19, a specific binding agent to CD3, and IL-12 and optionally a pharmaceutically acceptable excipient. In certain embodiments, the pharmaceutical composition is in the form of a powder in a vial. In certain embodiments, the pharmaceutical composition is in the form of a pH buffered saline solution.

In certain embodiments, the nanoparticle comprises on the surface a specific binding agent to an IL-12 sequence, wherein the specific binding agent to the IL-12 sequence is specifically binding IL-12.

In certain embodiments, the nanoparticle comprises on the surface a specific binding agent to an antigen sequence, wherein the specific binding agent to the antigen is specifically binding the antigen sequence conjugated to IL-12 comprising the antigen.

In certain embodiments, the nanoparticle has a hydrodynamic diameter of less than 200, 150 or 100 nm and comprises less than 5 specific binding agents to CD19 on the surface of the particle. In certain embodiments, the nanoparticle has a hydrodynamic diameter of less than 200, 150 or 100 nm and comprises between 20 to 70 specific binding agents to CD3 on the surface of the particle.

In certain embodiments, the nanoparticle has a ratio of specific binding agents to CD3 compared to specific binding agents to CD19 that is in excess of 10-fold or 20-fold.

In certain embodiments, this disclosure relates to pharmaceutical compositions comprising a) a molecular arrangement comprising a specific binding agent to CD19 conjugated to a specific binding agent to CD3; b) IL-12, and c) an optional pharmaceutically acceptable excipient. In certain embodiments, the pharmaceutical composition is in the form of a powder in a vial. In certain embodiments, the pharmaceutical composition is in the form of a pH buffered saline solution.

In certain embodiments, this disclosure relates to pharmaceutical compositions comprising a) a molecular arrangement comprising IL-12 conjugated to a multispecific immunoglobulin construct comprising a specific binding agent to CD19 conjugated to a specific binding agent to CD3 and b) optionally pharmaceutically acceptable excipient. In certain embodiments, the pharmaceutical composition is in the form of a powder in a vial. In certain embodiments, the pharmaceutical composition is in the form of a pH buffered saline solution.

Also provided herein are pharmaceutical compositions that includes molecular arrangements, nanoparticles, polypeptides, vectors, and nucleic acids as disclosed herein, together with a pharmaceutically acceptable excipient, such as, for example, a diluent or carrier. Agents and pharmaceutical compositions suitable for use in the present disclosure include those wherein the agent can be administered in an effective amount to achieve its intended purpose.

Suitable pharmaceutical formulations can be determined by the skilled artisan depending on the route of administration and the desired dosage. See, e.g., Remington's Pharmaceutical Sciences, 1435-712 (18th ed., Mack Publishing Co, Easton, Pa., 1990). Formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein as well as the pharmacokinetic data obtainable through animal or human clinical trials.

The phrases “pharmaceutically acceptable” or “pharmacologically acceptable” refer to molecular entities and compositions that do not produce adverse, allergic, or other untoward reactions when administered to an animal or a human. As used herein, “pharmaceutically acceptable excipients” includes any and all solvents, dispersion media, coatings, antibacterial and antifungal agents, isotonic and absorption delaying agents and the like. The use of such excipients for pharmaceutically active substances is well known in the art. Except insofar as any conventional media or agent is incompatible with the therapeutic compositions, its use in therapeutic compositions is contemplated.

The agents can be present in a pharmaceutical composition as a pharmaceutically acceptable salt. As used herein, “pharmaceutically acceptable salts” include, for example base addition salts and acid addition salts.

Pharmaceutically acceptable base addition salts may be formed with metals or amines, such as alkali and alkaline earth metals or organic amines. Pharmaceutically acceptable salts of compounds may also be prepared with a pharmaceutically acceptable cation. Suitable pharmaceutically acceptable cations are well known to those skilled in the art and include alkaline, alkaline earth, ammonium, and quaternary ammonium cations. Carbonates or hydrogen carbonates are also possible. Examples of metals used as cations are sodium, potassium, magnesium, ammonium, calcium, or ferric, and the like.

For oral administration, suitable compositions can be formulated readily by combining an agent disclosed herein with pharmaceutically acceptable excipients such as carriers well known in the art. Such excipients and carriers enable the present compounds to be formulated as tablets, pills, capsules, liquids, gels, syrups, slurries, suspensions, and the like, for oral ingestion by a patient to be treated. Pharmaceutical preparations for oral use can be obtained by adding a compound as disclosed herein with a solid excipient, optionally grinding a resulting mixture, and processing the mixture of granules, after adding suitable auxiliaries, if desired, to obtain tablets. Suitable excipients include, for example, fillers and cellulose preparations. If desired, disintegrating agents can be added. Pharmaceutically acceptable ingredients are well known for the various types of formulation and may be for example binders (e.g., natural or synthetic polymers), lubricants, surfactants, sweetening and flavoring agents, coating materials, preservatives, dyes, thickeners, adjuvants, antimicrobial agents, antioxidants and carriers for the various formulation types.

When a therapeutically effective amount of a compound disclosed herein is administered orally, the composition typically is in the form of a solid (e.g., tablet, capsule, pill, powder, or troche) or a liquid formulation (e.g., aqueous suspension, solution, elixir, or syrup).

When administered in liquid or suspension form, a functional liquid and/or a liquid carrier such as water, petroleum, or oils of animal or plant origin can be added. The liquid form of the composition can further contain physiological saline solution, sugar alcohol solutions, dextrose or other saccharide solutions, or glycols. For administration in liquid form, the composition may be supplied as a rapidly-dissolving solid formulation for dissolution or suspension immediately prior to administration.

When a therapeutically effective amount of a compound disclosed herein is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, in addition to a compound disclosed herein, an isotonic vehicle. Such compositions may be prepared for administration as solutions of free base or pharmacologically acceptable salts in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations can optionally contain a preservative to prevent the growth of microorganisms.

Injectable compositions can include sterile aqueous solutions, suspensions, or dispersions and sterile powders for the extemporaneous preparation of sterile injectable solutions, suspensions, or dispersions. In certain embodiments, the form is sterile and fluid, e.g., can be a solvent or dispersion medium containing, for example, water, ethanol, polyol (e.g., glycerol, propylene glycol, and liquid polyethylene glycol, and the like), suitable mixtures thereof, and vegetable oils. In one embodiment contemplated, the carrier is non-aqueous or substantially non-aqueous.

The prevention of the action of microorganisms can be brought about by various antibacterial and antifungal agents, for example, parabens, chlorobutanol, phenol, sorbic acid, thimerosal, and the like. In certain embodiments, it will be preferable to include isotonic agents, for example, sugars or sodium chloride.

Sterile injectable solutions can be prepared by incorporating the active agent in the required amount in the appropriate solvent with various of the other ingredients enumerated above, as required, followed by filtered sterilization.

### Examples

### Rapid Assembly and Screening of Multivalent Immune Cell-Redirecting Therapies for Leukemia

Immune cell redirection (ICR) is a powerful and versatile therapeutic approach in which the cytotoxic activity of endogenous immune cells is redirected toward diseased cells via simultaneous, drug-induced cell binding. This strategy has demonstrated potential therapeutic benefits in preclinical models of cancer, HIV, lupus, and other diseases. ICR therapies can potentially to co-opt a wide range of cell types (e.g., T cells, NK cells, and macrophages) against both cell-surface and intracellular targets. ICR immunotherapies can also vary widely in their composition and mode of delivery. They encompass nanoparticle, bispecific IgG, scFv fusion, and mRNA constructs, as well as vectors based on oncolytic viruses and engineered cells.

Reported herein is a method for the rapid assembly and screening of multivalent ICR drug candidates that redirect the lytic activity of T cells toward leukemic B cells and simultaneously codeliver T cell stimulating IL-12 to yield multifunctional therapies referred to as, bispecific T cell-engaging cytokines (BiTEokines). Using this discovery platform, cytokine codelivery can dramatically alter the antileukemic activity of ICR immunotherapies and the generation and screening of diverse libraries of BiTEokine candidates can be achieved. Extensions of this approach enable the rapid identification of drug compounds with activity against cancer, autoimmune diseases, or pathogen infections and, given its modular nature, could be extended to a wide range of immune cells, diseased cells, and soluble protein combinations.

Compositionally diverse libraries of these CD19×CD3×IL12 BiTEokines were assembled and screened enabling rapid hit identification and the delineation of important composition-function relationships. Using this approach, BiTEokine hit compounds were identified which exhibit ex vivo lytic activity comparable to therapies for leukemia. Detailed analysis of BiTEokine activity strongly correlated drug treatment with specific cell—cell contact, IL-12 delivery, and leukemia cell lysis. These results are particularly promising given that use as an in vivo therapeutic benefit from IL-12, due to immune memory resulting from cross-exposure of cytokine with antigens from lysed target cells. Optimal antibody composition and density can be rapidly determined using this approach; such molecular arrangements can be applied to structurally analogous antibody-conjugated liposomes, virus-like particles (VLPs), and self-assembled protein cages. Future studies investigating the impact of core scaffold size, spatial ordering antibodies, targeted cytokine neutralization, rather than delivery, may also lead to further improvements in BiTEokine activity or an expansion of disease targets, respectively. Given the rapid and modular nature of the approach presented here, facile extension to a wide range of immune cells, diseased cells, and soluble protein combinations are contemplated.

### IL-12 Enhances Bispecific T Cell Engager Activity

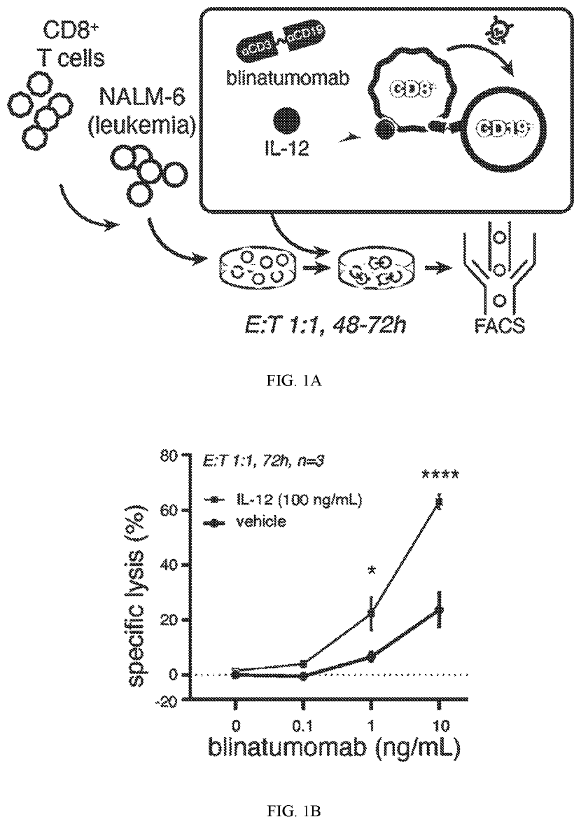

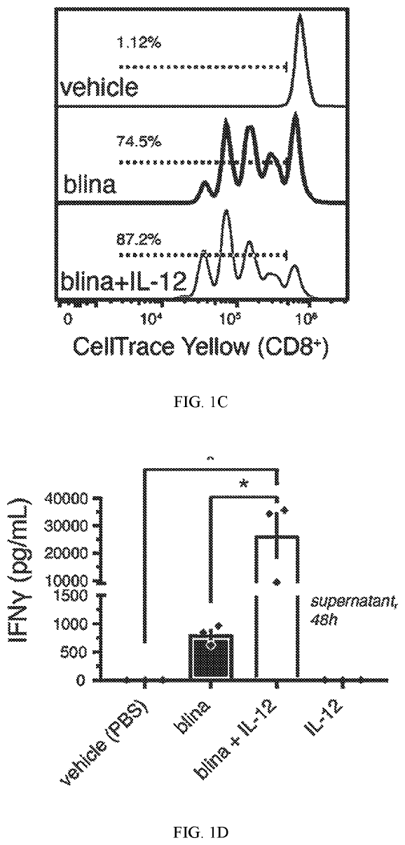

IL-12 can improve cancer immune elimination via enhanced CD8+ T cell proliferation, cytotoxicity, survival, and T cell receptor (TCR) signaling. IL-12 may improve the lytic activity of blinatumomab which bispecifically targets T cell CD3 and leukemic B cell CD19. Following prolonged coculture of primary CD8+ T cells with CD19+ NALM-6 leukemia cells, significant improvement in target leukemia cell lysis in the presence of IL-12 was observed as measured by flow cytometry, as well as an associated increase in T cell proliferation (FIG. 1C), and T cell activation (FIG. 1D), as measured by dye dilution and interferon gamma (IFNγ) secretion, respectively. Together, these data indicate that IL-12 can improve the performance of T cell redirecting therapies in an ex vivo assay that is heavily relied upon to prioritize ICR drug candidates and that these effects are attributable, in part, to cytokine-enhanced T cell proliferation and activation.

This marked effect of IL-12 on blinatumomab activity is significant in that other T cell mitogens such as IL-2 have been previously combined with blinatumomab with relatively little impact on lytic activity. While the synergy observed here may be unique to IL-12, such differential effects may arise due to the fact that typical lysis assays are performed over much shorter durations (e.g., 4 h) and that the effects of IL-12 result, in part, due to cytokine-induced T cell proliferation (P=0.020, FIG. 1C). Interestingly, however, T cell expansion observed in the presence of both drugs was, alone, insufficient to fully account for the large change in target cell lysis.

Synergy between blinatumomab and recombinant IL-12 is significant in that the former is currently approved to treat relapsed/refractory and minimal residual disease positive (MRD+) B-ALL in both adults in children. And while IL-12 therapies have not advanced to phase III trials due to poor circulation and toxicity, several IL-12 drug candidates currently under investigation may benefit from combination with blinatumomab including adenoviral, plasmid, mRNA, and affinity-targeted IL-12.

### BiTEokine Synthesis and Characterization

Magnetic nanoparticles (50-80 nm) functionalized with protein G were obtained from a commercial supplier. Particles were validated for lot-specific sizing via DLS. BiTEokine test compounds were prepared via addition of 1.65×10¹¹ particles to antibody mixtures (PBS) for 10 min at room temperature with agitation at 200 rpm. Antibody amounts per well ranged from 0.11 μg to 2.28 μg (αCD19), 1.25 μg to 24.31 μg (αCD3), 0.91 μg to 8.38 μg (αIL12), 1.76 μg to 11.27 μg (Isotype). Unbound antibodies were removed via magnetic field-induced sedimentation (>8 min) and washing twice with PBS. Previously obtained calibration curves for antibody-particle binding were used to establish conditions for compound library preparation. Intermediate compounds were then passed through 0.45 μm sterile PVDF filters and 2 eq. of recombinant human single chain IL-12 (relative to αIL12 binding sites) was added to a subset of particles for 30 min at room temperature with agitation at 200 rpm. Purified test compounds were obtained after magnetic field-induced sedimentation (>8 min) and washing twice with PBS. Antibody abundance on test compounds was determined from spillover-corrected fluorescence intensity and comparison to standard curves for each fluorochrome-conjugated antibody.

### Design and Rapid Screening of CD19×CD3×IL12 BiTEokines

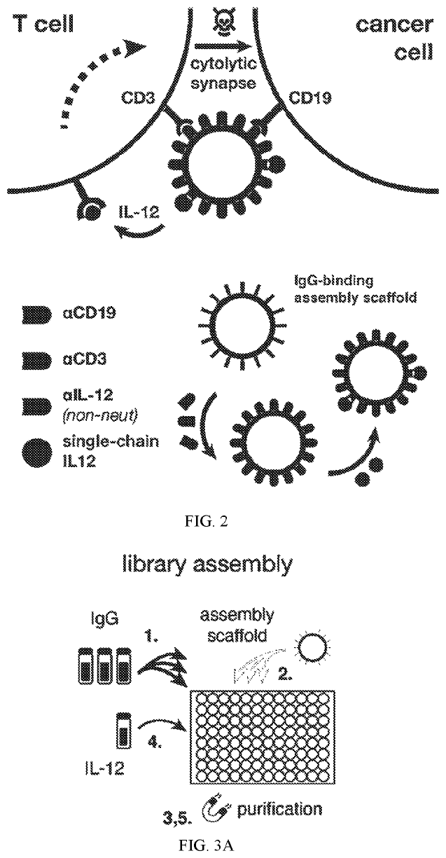

Having shown that IL-12 potentiates the antileukemic activity of T cell-redirecting immunotherapy, a drug architecture was designed that (i) directs the lytic activity of T cells toward leukemic B cells, (ii) simultaneously codelivers T cell-stimulating IL-12, and (iii) features a modular design amenable to combinatorial assembly of test compound libraries (FIG. 2). the core scaffold of these structures was based on magnetic iron oxide nanoparticles due to ability to accommodate a wide range of IgG antibodies via Fc-protein G affinity, and rapid purification via magnetic field sedimentation. Antibody clones were selected due to their prior clinical testing as CAR-T cell constructs (CD19, SJ25-C1) or antibody-drug conjugates (CD3, UCHT1), and their comparable IgG1-protein G affinity. Using this modular design, varying BiTEokine protein abundance results in differential capacity for drug-induced leukemia cell lysis through altered cytokine concentration or affinity/avidity toward cell-surface epitopes.

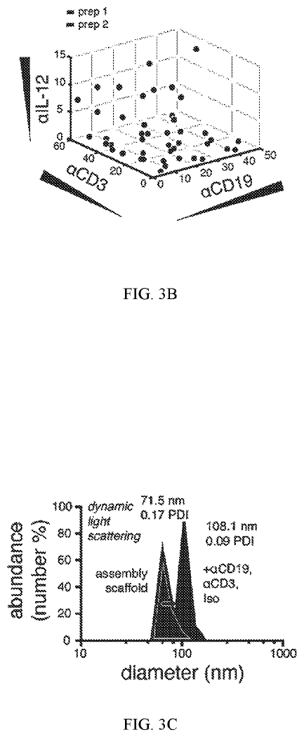

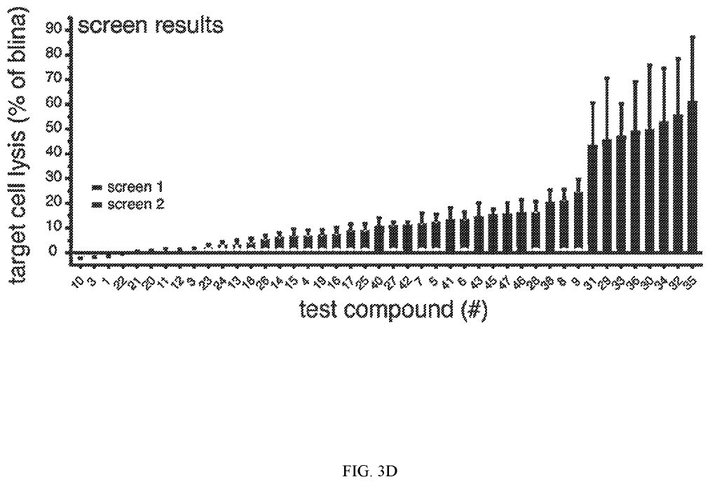

To assemble BiTEokine test compound libraries, equivalent numbers of magnetic nanoparticles were dispensed into individual wells of a standard 96 well plate, each containing varying cocktails of fluorochrome-labeled antibodies directed against human CD19, CD3ε, (non-neutralizing) IL-12, or isotype control (FIG. 3A). After incubation, magnetic field-induced sedimentation, and analysis of antibody abundance using a standard fluorescence plate reader, the beads were sterile-filtered and varying volumes of a single-chain variant of IL-12 (scIL-12) were added followed by incubation and further purification. Test compounds prepared using this method were highly reproducible batch-to-batch, exhibiting stable hydrodynamic size and consistent antibody composition. Using this approach, BiTEokine libraries were prepared and characterized over the course of just 8 to 9 h.

In total, 47 unique BiTEokine test compounds were synthesized which varied widely in antibody composition, achieving a consistent, and near theoretical maximum, total coverage of 134±15 IgG per particle (αCD19:1.5±0.8 to 42±4; αCD3:2.5±0.5 to 59±3; αIL12 0.12±0.08 to 15±0.7; FIG. 3B). Dynamic light scattering measurements indicated high stability of the subsequent test compounds in buffer, with hydrodynamic size increasing from 71.5 to 108.1 nm upon antibody surface-assembly (FIG. 3C) with no appreciable change in particle morphology as measured by transmission electron microscopy. Composition of the CD19×CD3×IL12 BiTEokine test compound library was measured by antibody fluorescence intensity.

TABLE 1

| | Antibodies per bead | | | | | | | | | | | | |
|---|---|---|---|---|---|---|---|---|---|---|---|---|---|
| | aCD3 | | | aCD19 | | | aIL-12 | | | Isotype | | | |
| | (UCHT1) | | | (SJ25-C1) | | | (C11.5) | | | (MOPC21) | | | |
| BiTEokine | Mean | SD | N | Mean | SD | N | Mean | SD | N | Mean | SD | N | Ab/bead |
| 1 | 2.49 | 0.47 | 3 | 5.307 | 1.08 | 3 | 0.24 | 0.05 | 3 | 125.4 | 20.3 | 3 | 133.476 |
| 2 | 3.153 | 0.48 | 3 | 7.73 | 3.80 | 3 | 0.903 | 0.06 | 3 | 157.7 | 35.4 | 3 | 169.523 |
| 3 | 3.237 | 0.53 | 3 | 7.023 | 0.70 | 3 | 1.887 | 0.14 | 3 | 134 | 15.4 | 3 | 146.153 |
| 4 | 19.41 | 2.52 | 3 | 8.087 | 2.22 | 3 | 0.23 | 0.09 | 3 | 147.4 | 37.8 | 3 | 175.136 |
| 5 | 17.37 | 0.64 | 3 | 5.617 | 2.05 | 3 | 0.773 | 0.096 | 3 | 122.5 | 11.5 | 3 | 146.263 |
| 6 | 18.63 | 2.41 | 3 | 7.307 | 2.16 | 3 | 1.533 | 0.076 | 3 | 118.5 | 19.4 | 3 | 145.98 |
| 7 | 38.06 | 1.11 | 3 | 5.363 | 1.86 | 3 | 0.123 | 0.076 | 3 | 98.93 | 6.25 | 3 | 142.473 |
| 8 | 40.17 | 2.62 | 3 | 6.057 | 1.34 | 3 | 0.743 | 0.015 | 3 | 103.1 | 12.0 | 3 | 150.096 |
| 9 | 43.39 | 1.45 | 3 | 5.957 | 1.17 | 3 | 1.47 | 0.104 | 3 | 101.8 | 8.77 | 3 | 152.593 |
| 10 | 3.593 | 0.14 | 3 | 25.47 | 3.36 | 3 | 0.237 | 0.038 | 3 | 128.6 | 16.9 | 3 | 157.893 |
| 11 | 3.64 | 1.1 | 3 | 21.62 | 3.28 | 3 | 0.817 | 0.021 | 3 | 115.2 | 12.9 | 3 | 141.273 |
| 12 | 3.05 | 0.38 | 3 | 14.16 | 2.46 | 3 | 1.627 | 0.023 | 3 | 85.68 | 3.84 | 3 | 104.52 |
| 13 | 20.75 | 2.67 | 3 | 21.52 | 8.93 | 3 | 0.17 | 0.066 | 3 | 105.3 | 12.2 | 3 | 147.753 |
| 14 | 19.77 | 1.23 | 3 | 22.42 | 3.50 | 3 | 0.643 | 0.142 | 3 | 92.7 | 12.8 | 3 | 135.536 |
| 15 | 19.19 | 1.52 | 3 | 24.34 | 2.74 | 3 | 1.52 | 0.036 | 3 | 91.55 | 5.79 | 3 | 136.6 |
| 16 | 40.38 | 2.53 | 3 | 20.89 | 3.09 | 3 | 0.153 | 0.021 | 3 | 81.21 | 7.65 | 3 | 142.636 |
| 17 | 41.77 | 2.29 | 3 | 24.32 | 1.07 | 3 | 0.63 | 0.044 | 3 | 83.69 | 9.55 | 3 | 150.406 |
| 18 | 39.47 | 2.32 | 3 | 20 | 6.55 | 3 | 1.377 | 0.055 | 3 | 69.68 | 6.18 | 3 | 130.526 |
| 19 | 22.94 | 0.58 | 3 | 22.1 | 4.67 | 3 | 0.927 | 0.006 | 3 | 97.58 | 5.91 | 3 | 143.55 |
| 20 | 4.067 | 0.19 | 3 | 41.06 | 3.19 | 3 | 0.26 | 0.052 | 3 | 101.3 | 9.51 | 3 | 146.686 |
| 21 | 3.687 | 0.8 | 3 | 37.07 | 6.56 | 3 | 0.85 | 0.098 | 3 | 91.47 | 13.9 | 3 | 133.073 |
| 22 | 3.39 | 0.21 | 3 | 42.04 | 12.7 | 3 | 1.77 | 0.053 | 3 | 82.07 | 12.3 | 3 | 129.263 |
| 23 | 17.66 | 1.1 | 3 | 36.76 | 4.42 | 3 | 0.237 | 0.055 | 3 | 72.84 | 9.19 | 3 | 127.50 |
| 24 | 17.78 | 0.57 | 3 | 38.52 | 1.15 | 3 | 0.743 | 0.042 | 3 | 69.88 | 3.23 | 3 | 126.92 |
| 25 | 21.05 | 1.27 | 3 | 37.89 | 7.48 | 3 | 1.673 | 0.057 | 3 | 73.04 | 4.34 | 3 | 133.65 |
| 26 | 37.56 | 1.62 | 3 | 36.93 | 5.97 | 3 | 0.173 | 0.031 | 3 | 63.05 | 4.50 | 3 | 137.716 |
| 27 | 37.06 | 3.31 | 3 | 30.4 | 1.28 | 3 | 0.667 | 0.031 | 3 | 51.98 | 5.18 | 3 | 120.106 |
| 28 | 48.57 | 2.05 | 3 | 40.05 | 5.00 | 3 | 1.5 | 0.07 | 3 | 58.21 | 3.63 | 3 | 148.326 |
| 29 | 24.42 | 0.6 | 3 | 1.517 | 0.82 | 3 | 1.42 | 0.089 | 3 | 75.26 | 7.30 | 3 | 102.61 |
| 30 | 26.28 | 1.24 | 3 | 2.677 | 0.64 | 3 | 7.707 | 0.505 | 3 | 78.36 | 7.31 | 3 | 115.03 |
| 31 | 29.56 | 1.21 | 3 | 3.107 | 1.92 | 3 | 12.79 | 0.747 | 3 | 78.95 | 10.9 | 3 | 124.40 |
| 32 | 39.18 | 3.5 | 3 | 3.067 | 0.80 | 3 | 1.313 | 0.137 | 3 | 81.66 | 16.1 | 3 | 125.22 |
| 33 | 43.25 | 2.04 | 3 | 1.75 | 0.29 | 3 | 6.807 | 0.284 | 3 | 75.25 | 9.44 | 3 | 127.05 |
| 34 | 44.04 | 0.78 | 3 | 2.207 | 1.58 | 3 | 11.21 | 0.556 | 3 | 64.11 | 2.73 | 3 | 121.56 |
| 35 | 51.36 | 4.93 | 3 | 3.163 | 0.30 | 3 | 1.24 | 0.095 | 3 | 71.97 | 12.0 | 3 | 127.73 |
| 36 | 58.92 | 3.08 | 3 | 2.497 | 1.19 | 3 | 7.143 | 0.214 | 3 | 66 | 1 | 3 | 134.55 |
| 37 | 55.25 | 5.37 | 3 | 2.377 | 0.64 | 3 | 10.65 | 0.381 | 3 | 48.03 | 4.96 | 3 | 116.30 |
| 38 | 41.01 | 2.57 | 3 | 21.54 | 2.70 | 3 | 7.893 | 0.375 | 3 | 59.83 | 2.85 | 3 | 130.28 |
| 39 | 29.9 | 1.74 | 3 | 40.37 | 2.79 | 3 | 1.343 | 0.045 | 3 | 63.35 | 3.89 | 3 | 134.97 |
| 40 | 29.03 | 1.87 | 3 | 31.69 | 1.04 | 3 | 7.977 | 0.179 | 3 | 50.81 | 1.19 | 3 | 119.51 |
| 41 | 34.53 | 2.79 | 3 | 42.34 | 4.19 | 3 | 15 | 0.718 | 3 | 51.57 | 2.32 | 3 | 143.43 |
| 42 | 44.8 | 1.69 | 3 | 38.56 | 2.60 | 3 | 1.307 | 0.136 | 3 | 46.32 | 3.30 | 3 | 130.986 |
| 43 | 48.22 | 0.98 | 3 | 39.71 | 6.15 | 3 | 6.753 | 0.292 | 3 | 39.27 | 1.95 | 3 | 133.95 |
| 44 | 44.57 | 2.93 | 3 | 37.49 | 3.11 | 3 | 11.88 | 0.283 | 3 | 31.6 | 2.55 | 3 | 125.54 |
| 45 | 49.25 | 1.58 | 3 | 27.41 | 5.45 | 3 | 1.077 | 0.097 | 3 | 25.52 | 2.36 | 3 | 103.256 |
| 46 | 53.96 | 2.02 | 3 | 34.86 | 1.98 | 3 | 6.107 | 0.076 | 3 | 24.46 | 0.94 | 3 | 119.39 |
| 47 | 54.6 | 1.67 | 3 | 33.27 | 5.46 | 3 | 11.03 | 0.287 | 3 | 18.9 | 2 | 3 | 117.796 |

Size increases (ca. 36.5 nm) correspond closely to what one would expect following addition of a single monolayer of IgG1 (10-12 nm hydrodynamic diameter) about these particles, augmenting their size to well-above the size threshold for renal clearance in humans, thus potentially improving the circulation and associated therapeutic benefit of IL-12 therapy both alone and in combination with ICR immunotherapy. Together these data demonstrate that BiTEokine test compound libraries can be assembled rapidly, in parallel with a wide range of structural diversity.

With a test compound library in-hand, the lytic activity of BiTEokines were screened following incubation with cocultures of primary human CD8+ T cells and CD19+ NALM-6 leukemia cells and analysis by flow cytometry (FIG. 3D). These screens were performed over 72 h using a low effector-to-target (E:T) ratio of 1:1 to allow for observable T cell proliferation and to closely reflect cell counts present in the peripheral blood of patients with MRD+ BALL and post-transplant relapsed B-ALL, populations for whom blinatumomab therapy is currently FDA-approved. BiTEokine test compounds varied widely in their corresponding lytic potential with median activity just 11% (n=3) that of blinatumomab's. The top screening hits, 30, 32, 34-36, in contrast, exhibited lytic activity closely comparable to, and statistically indistinguishable (P>0.98) from, blinatumomab (e.g., 035:62±26%). Interestingly, these top performing BiTEokines displayed only a small number of B cell-targeting antibodies per particle (2±1 to 3.2±0.3) and an abundance for CD3 antibodies (24±1 to 59±3), thus largely limiting the potential for interaction with multiple B cells.

An attractive feature of this combinatorial screening approach is its ability to rapidly shed light on composition function relationships unique to these novel multivalent drug architectures. For example, the bispecific antibody, blinatumomab, targets T and leukemic B cells with low and high affinity, respectively. Multivariate least-squares regression modeling of screening data (R2=0.82) indicated significant contributions from all BiTEokine antibody components; however surprisingly, here we observed that high αCD3/αCD19 ratio was, in fact, more closely associated with favorable lytic activity. This effect is surprising based on disparate antigen ratio (approx. 1.2:1.0 CD19:CD3) or density given that this cell line was used extensively in the preclinical development of blinatumomab. The nonobvious finding that optimally lytic structures displayed effector cell antibodies 10- to 25-fold in excess of those toward target cells is significant in that prior studies of other multivalent ICR immunotherapies focus on either single (i.e., approximately equimolar) or narrow ranges of antibody composition (e.g., 0.33- to 3-fold). Antibody ratios in these ranges induced only low levels of leukemia cell lysis in the experiments; thus, the already impressive performance of prior multivalent ICR immunotherapies may be further improved by the systematic discovery approach described here. Anti-(α)CD19 abundance was a negative correlate of lytic activity and, conversely, αCD3, αIL12, and T cell division (% divided) were positive correlates of leukemia cell lysis.

Target cell lysis was also nominally improved by the tethering of IL-12 to BiTEokines in comparison to coadministration of cytokine with αIL12-deficient analogs. Interestingly, the impact of IL-12 on drug activity appeared to bifurcate depending on its relative abundance on BiTEokines with low amounts of cytokine (approximately 2.3 ng/mL) inducing high T cell division but low leukemia cell lysis, and high IL-12 (approximately 270 ng/mL) inducing less rapid T cell division and high target cell lysis. The ability of multivalent BiTEokines to induce TCR clustering may allow them to redirect the activity of CD8+ T cell subsets outside of those typically acted upon by bispecific antibodies and (ii) IL-12 concentration-dependent CD8+ T cell differentiation may enrich for T cell subsets with differential dependency on costimulation for drug-induced lysis (e.g., memory precursor or short-lived effector cells).

### Activity and Specificity of BiTEokines

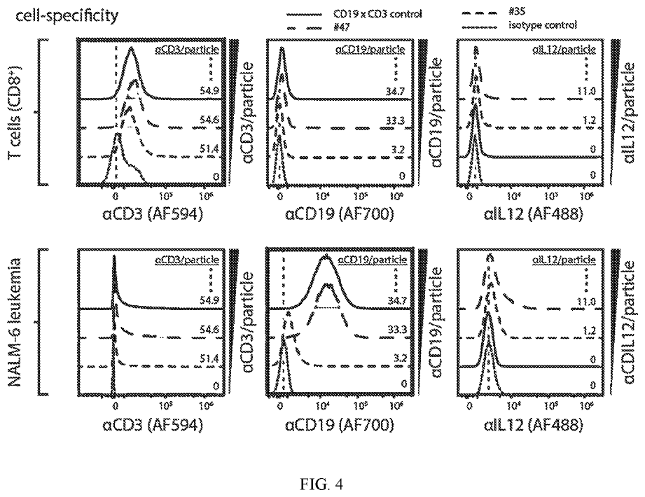

After identifying hits from the BiTEokine activity screen, experiments were performed to determine whether these compounds specifically targeted T and leukemic B cells via flow cytometry. Particle abundance-dependent increases were observed in the labeling of T cells with CD3 antibodies and B cells with CD19 antibodies, but no apparent αIL12-dependent cell specificity from BiTEokine test compounds (FIG. 4). In addition, BiTEokine lytic activity arose from precise combinations of antibodies, rather than nonspecific antibody interactions, using compounds either fully or partially conjugated with isotype control antibody to approximately equivalent total amounts of IgG. While isotype control BiTEokines elicited only basal levels of activity (1.9% lysis), greater than 17-fold increases in lytic responses we observed from lead compounds 35 and 37. Together, these data indicate cell-specific binding by BiTEokines with the lysis of leukemic B cells.

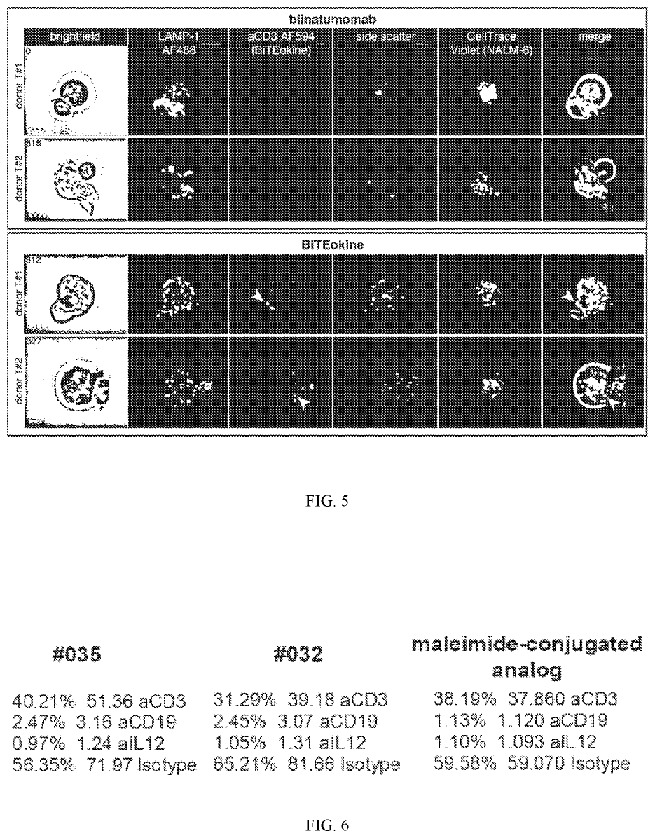

To further characterize the role of BiTEokines in inducing leukemia cell lysis, imaging flow cytometry experiments were performed on cocultures of primary human CD8+ T cells and CD19+ NALM-6 leukemia cells treated with fluorescently labeled hit compound 35 from the prior activity screen, as well as blinatumomab. Gating on doublets of T and B cells, similar patterns of LAMP-1 (CD107a) positive vesicle accumulation, indicative of lytic granules and lysosomes, were observed in both blinatumomab- and BiTEokine-treated cocultures (FIG. 5). Strikingly, distinct accumulation of BiTEokines were observed at the interface between T and leukemic B cells, further associating BiTEokine treatment with cell-cell contact with leukemia cell lysis.

Analogs in which Antibodies are Bound to the Particle Surfaces Using Covalent Chemistry

Lyophilized, maleimide functionalized 50-80 nm iron oxide nanoparticles (Ocean Nanotech) were resuspended at 6e11 particles/mL in degassed PBS (1 mM EDTA, 0.01% Tween20, 5% DMSO). Antibodies were reduced with 0.01-0.1 mM TCEP for 60 minutes at room temperature in degassed PBS (1 mM EDTA). Following reduction, antibody mixtures are immediately reacted with maleimide nanoparticles overnight at 4° C. using end-over-end mixing in degassed PBS (1 mM EDTA, 0.01% Tween20, 5% DMSO). Unbound antibodies were removed with the supernatant following magnetic sedimentation. Antibody abundance on the purified nanoparticles was characterized using spillover-corrected fluorescence intensity. Following sterile 0.45 micrometer syringe filtration, IL-12 was bound to the nanoparticles following 30 minute incubation at room temperature using a 2× saturating amount of IL-12 per nanoparticle. Excess reactants and reaction buffer components were removed by magnetic sedimentation with resuspension of the final product in sterile PBS and storage at 4° C. Flow cytometry dot plots of B- and T-cell cocultures showed specific labeling of B cells and T cells with maleimide-conjugated BiTEokine analogs.

What is claimed is:

1. A method of treating an immune regulated disease comprising administering to a subject in need thereof an effective amount of a grouped molecular arrangement comprising a specific binding agent to CD19, a specific binding agent to CD3, and interleukin-12.

2. The method of claim 1, wherein the immune regulated disease is cancer, chronic viral infection, or lupus.

3. A method of treating an immune regulated disease comprising administering to a subject in need thereof an effective amount of a nanoparticle comprising on the surface of the nanoparticle a specific binding agent to CD19, a specific binding agent to CD3, and interleukin-12.

4. The method of claim 3, wherein the immune regulated disease is cancer, chronic viral infection, or lupus.

5. The method of claim 3, wherein the nanoparticle has a hydrodynamic diameter of less than 200 nm and comprises less than 5 specific binding agents to CD19 on the surface of the particle.

6. The method of claim 3, wherein the nanoparticle has a hydrodynamic diameter of less than 200 nm and comprises between 20 to 70 specific binding agents to CD3 on the surface of the particle.

7. The method of claim 3, wherein the nanoparticle has a ratio of specific binding agents to CD3 compared to specific binding agents to CD19 that is in excess of 10-fold.

8. The method of claim 3, wherein the individual nanoparticles have a hydrodynamic size of less than 200 nm, have 25 to 60 specific binding agents to CD3, 2 to 4 specific binding agents to CD19, and 1 to 12 interleukin-12 sequences on the surface of the nanoparticle.

9. The method of claim 8, wherein the specific binding agents to CD3 are antibodies.

10. The method of claim 8, wherein the specific binding agents to CD19 are antibodies.

11. A pharmaceutical composition comprising a nanoparticle comprising on the surface of the nanoparticle a specific binding agent to CD19, a specific binding agent to CD3, and interleukin-12.

12. The pharmaceutical composition of claim 11, wherein the nanoparticle comprises on the surface a specific binding agent to an IL-12 sequence, and wherein the specific binding agent to the IL-12 is specifically binding IL-12.

13. The pharmaceutical composition of claim 11, wherein the nanoparticle comprises on the surface a specific binding agent to an antigen sequence, and wherein the specific binding agent to the antigen is specifically binding the antigen sequence conjugated to IL-12 comprising the antigen.

14. The pharmaceutical composition of claim 11, wherein the nanoparticle has a hydrodynamic diameter of less than 200 nm and comprises less than 5 specific binding agents to CD19 on the surface of the particle.

15. The pharmaceutical composition of claim 11, wherein the nanoparticle has a hydrodynamic diameter of less than 200 nm and comprises between 20 to 70 specific binding agents to CD3 on the surface of the particle.

16. The pharmaceutical composition of claim 11, wherein the nanoparticle has a ratio of specific binding agents to CD3 compared to specific binding agents to CD19 that is in excess of 10-fold.

17. The pharmaceutical composition of claim 11, wherein the nanoparticle has a ratio of specific binding agents to CD3 compared to specific binding agents to CD19 that is in excess of 20-fold.

18-20. (canceled)
